(19) United States
(12) Patent Application Publication
(10) Pub. No.: US 2011/0319728 A1
(43) Pub. Date: Dec. 29, 2011

(54) BLOOD PARAMETER SENSOR AND FLOW CONTROL SYSTEM, METHOD AND COMPUTER PROGRAM PRODUCT

(75) Inventors: James Petisce, San Clemente, CA (US); Emily Jackson, Mission Viejo, CA (US); Gregory J. Carlin, Atlanta, GA (US)

(73) Assignee: Edwards Lifesciences Corporation, Irvine, CA (US)

(21) Appl. No.: 12/826,542

(22) Filed: Jun. 29, 2010

(51) Int. Cl. A61B 5/145

(52) U.S. Cl. 600/309

(57) ABSTRACT

A system is provided for sensing a blood parameter, such as an analyte, in a patient's vasculature. The system includes a vascular access device, a parameter sensor, a pressure sensor and a flow control device. The vascular access device supports the parameter sensor within the vasculature of the patient and includes an internal passageway for drawing samples onto and calibrating the parameter sensor. The pressure sensor is within the internal passageway. The flow control device modulates sample draw, calibration and flushing fluid flows to prevent or remove clots based on communication with the pressure sensor. Advantageously, the dynamic, adaptive modulation of flow reduces flushing and other cycle times while at the same time improving clot flushing. As another advantage, this enables reduction or elimination of the use of heparin and other anticoagulant components in the solution supplied for flushing and/or calibration.

### TECHNICAL FIELD

The present disclosure relates generally to systems for introduction of sensors into the blood stream for sensing blood parameters and, more particularly, a flow control system for a vascular access device for delivering the sensors to the blood stream.

### BACKGROUND

Determination of intra-venous measurements of selected blood parameters are a boon to treatment of patients by professional healthcare workers. This is especially true for patients in the critical care setting, as these patients can have unstable conditions that can change rapidly and need dynamic detection and response.

Glucose is a blood parameter that has received a great amount of interest under the theory that tight glycemic control (TGC) benefits patients by keeping their blood glucose within a narrow range. Although not all healthcare workers are adherents to TGC, some TGC studies have shown that maintenance of normal glycemic ranges with insulin infusions reduced mortality of patients in the intensive care unit. Also, studies have shown a decreased incidence of acute renal failure, septicemia and critical illness polyneuropathy of patients under TGC.

Typically, critical care patients have their glucose tracked by the regular use of finger-stick tests. Although it generates valuable information, this approach has several drawbacks. It's labor intensive because the healthcare provider must stop and lance the patient on a regular such as an hourly—basis. Even with such effort, the samples are relatively few and far between and thus don't track dynamic changes in glucose very well. Also, the glucose present in a finger-stick sample does not always correspond to an intravenous sample, the latter of which is a better indication of most aspects of the patient's condition.

One solution to this is to use a system that monitors intravenous blood more directly, such as by connection to an existing peripheral or central venous catheter. Such systems have the advantage of being able to sample or pull blood at a regular interval without the additional intervention of a healthcare worker. However, the natural tendency of blood causes these systems to clot, either blocking flow of blood through the catheter or collecting on the sensor. One way to overcome this problem is to supply a flushing solution that includes the use of heparin. Heparin is an anticoagulant that can break up or inhibit the formation of clots. However, heparin use has fallen out of favor recently due to highly publicized injuries to patients who have received incorrect dosages. Therefore, many healthcare institutions are avoiding or stopping entirely the use of heparin either in a fluid line or as a coating on disposable supplies, such as sensors and catheters.

There is a need, therefore, for continuous glucose monitoring systems that provide substantially continuous sampling of a patient's glucose level. There is also a need to avoid the use of heparin and other anticoagulant components in intravascular sensor components, while at the same time having those sensor components still operate normally in a blood environment.

### SUMMARY

The present invention overcomes the problems of the prior art by providing a system for sensing a blood parameter, such as an analyte, in a patient's vasculature. The system includes a vascular access device, a parameter sensor, a pressure sensor and a flow control device. The vascular access device supports the parameter sensor within the vasculature of the patient and includes an internal passageway for moving samples onto and calibrating the parameter sensor. The pressure sensor, which can dynamically detect increased resistance to flow by clots or other obstructions, is supported within the passageway. The flow control device modulates sample draw, calibration and flushing fluid flows to prevent or remove clots based on communication with the pressure sensor. Advantageously, the dynamic, adaptive modulation of flow reduces flushing and other cycle times while at the same time reducing clot formation and/or improving clot flushing. As another advantage, this enables reduction or elimination of the use of heparin and other anticoagulant components in the solution supplied for flushing and/or calibration.

In one embodiment, the present invention includes a system for sensing a parameter of blood in a patient's vasculature. The system includes a vascular access device, a parameter sensor, a pressure sensor and a flow control device. The vascular access device, such as a catheter, connects to the patient's vasculature and defines an internal fluid passageway for fluid communication with the blood. Supported by the vascular access device is the parameter sensor. Within the internal fluid passageway of the vascular access device is the pressure sensor. The flow control device is configured to adapt fluid flow within the internal fluid passageway based on communication with the pressure sensor.

In one embodiment, the flow control device is configured to draw blood into the internal fluid passageway to cover the parameter sensor and to flush a solution over the parameter sensor. These two steps can be dynamically adapted by the flow control device based on the feedback from the pressure sensor so as to remove clots or reduce the incidence of clotting. For example, the flow control device may be configured to adapt (e.g., decrease) the draw time based on an increased pressure signal. Alternatively, or in addition, the flow control device can adapt (e.g., increase) the flush time in response to an increased pressure signal. Also, the flow control device can increase the flow rate for the flushing solution or the blood draw in response to pressure measurements communicated from the pressure sensor. In another aspect, the flow control device may reverse a draw into a flush in response to pressure measurements communicated by the pressure sensor.

In another embodiment, the flow control device may be connected in communication with the parameter sensor. The flow control device can further adapt the fluid flow within the internal fluid passageway based on communication with the parameter sensor.

In another aspect, at least a portion of the parameter sensor may be positioned within the lumen of the vascular access device and the pressure sensor is therefore configured to measure the pressure on the parameter sensor. Thus, the parameter sensor, and the pressure sensor, may also be within a hub of the vascular access device wherein the hub defines a portion of the internal fluid passageway.

In another embodiment, the flow control device includes a pump, a reservoir and a controller. The reservoir is connected in fluid communication with the internal fluid passageway and its flow is controlled by the controller. Advantageously, a solution within the reservoir used for flushing may have reduced or eliminated anticoagulant components.

In yet another embodiment, the internal fluid passageway extends from the patient's vasculature and through to a pump of the flow control device and at least two pressure sensors are arrayed along the internal fluid passageway.

In another embodiment, the flow control device is configured to adapt fluid flow based on a pressure gradient communicated by the pressure sensor. Further, the flow control device may include a memory for storing pressures communicated by the pressure sensor. Using the stored information, the flow control device can adapt fluid flow based on the stored values, such as by following a contour of a curve formed using the stored pressure values.

In yet another embodiment, the present invention may include just the flow control device as it is configured to communicate with the pressure sensor and adapt fluid flow based on pressures communicated by the pressure sensor.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

FIG. 1A is a perspective view of a vascular access device of a system for sensing a blood parameter of one embodiment of the present invention;

FIG. 1B is an elevation view of the vascular access device of FIG. 1A;

FIG. 1C1 is a partial sectional view of a fluid coupler of the vascular access device of FIG. 1A;

FIG. 1C2 is a partial sectional view of the vascular access device of FIG. 1A revealing a blood parameter sensor and a pressure sensor of another embodiment of the present invention;

FIGS. 1D and 1E are cross-sectional views that together show the entire vascular access device of FIG. 1A;

FIG. 1E1 is an enlarged view of the end of the cross-section of FIG. 1E showing a pressure sensor of another embodiment of the present invention;

FIG. 2 is a perspective view of an entire blood parameter measurement system of an embodiment of the present invention;

FIG. 3 is a schematic of the system shown in FIG. 2;

FIGS. 4A-4C is a schematic of a flow control device of another embodiment of the present invention;

FIG. 5 is a flowchart of a method and computer program product of a blood parameter measurement system of another embodiment of the present invention;

FIG. 6 is a perspective view of a multi-lumen access device of another embodiment of the present invention; and

FIG. 7 is a partial sectional view of the multi-lumen access device of FIG. 6 showing a pressure sensor in a hub or backform.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

The term “blood parameter” means any useful characteristic measurement of blood, including mechanical, such as hemodynamic characteristics like cardiac output, end-diastolic volume and extra-vascular lung volume, and chemical, such as analyte measurements defined below.

The term “vascular access device” as used herein is a broad term, and is to be given its ordinary and customary meaning to a person of ordinary skill in the art {and are not to be limited to a special or customized meaning), and refers without limitation to any device that is in communication with the vascular system of a host. Vascular access devices include but are not limited to catheters, shunts, tubing, blood withdrawal devices and the like.

The term “catheter” as used herein is a broad term, and is to be given its ordinary and customary meaning to a person of ordinary skill in the art (and are not to be limited to a special or customized meaning), and refers without limitation to a tube that can be inserted into a host's body (e.g., cavity, duct or vessel). In some circumstances, catheters allow drainage or injection of fluids or access by medical instruments or devices. In some embodiments, a catheter is a thin, flexible tube (e.g., a “soft” catheter). In alternative embodiments, the catheter can be a larger, solid tube (e.g., a “hard” catheter). The term “cannula” is interchangeable with the term “catheter” herein.

The term “pump” as used herein is a broad term, and is to be given its ordinary and customary meaning to a person of ordinary skill in the art (and are not to be limited to a special or customized meaning), and refers without limitation to a device used, in whole or in part, to move liquids, or slurries.

The term “valve” as used herein is a broad term, and is to be given its ordinary and customary meaning to a person of ordinary skill in the art (and are not to be limited to a special or customized meaning), and refers without limitation to a device that regulates the flow of substances (either gases, fluidized solids, slurries, or liquids), for example, by opening, closing, or partially obstructing a passageway through which the substance flows. A pump can include aspects of a valve and vice versa.

The terms “processor module” and “microprocessor” as used herein are broad terms, and are to be given their ordinary and customary meaning to a person of ordinary skill in the art {and are not to be limited to a special or customized meaning), and refer without limitation to a computer system, state machine, processor, and the like designed to perform arithmetic or logic operations using logic circuitry that responds to and processes the basic instructions that drive a computer.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-.beta. hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S. hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptherialtetanus antitoxin; erythrocyte arginase; erythrocyte protoporphyrin; esterase D; fatty acids/acylglycines; free .beta.-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, .beta.); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

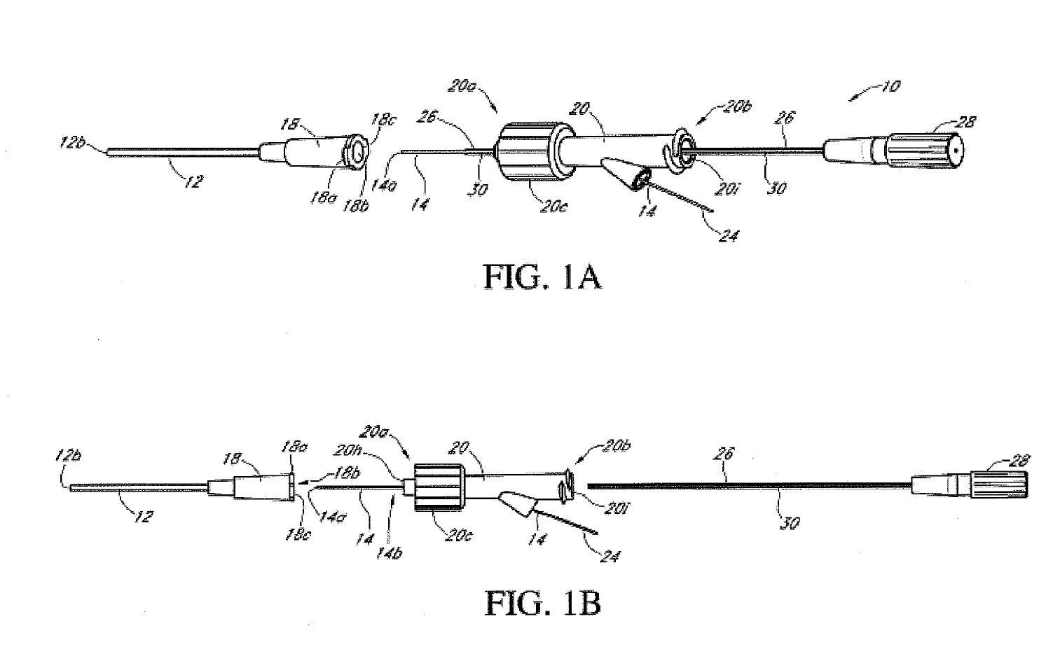

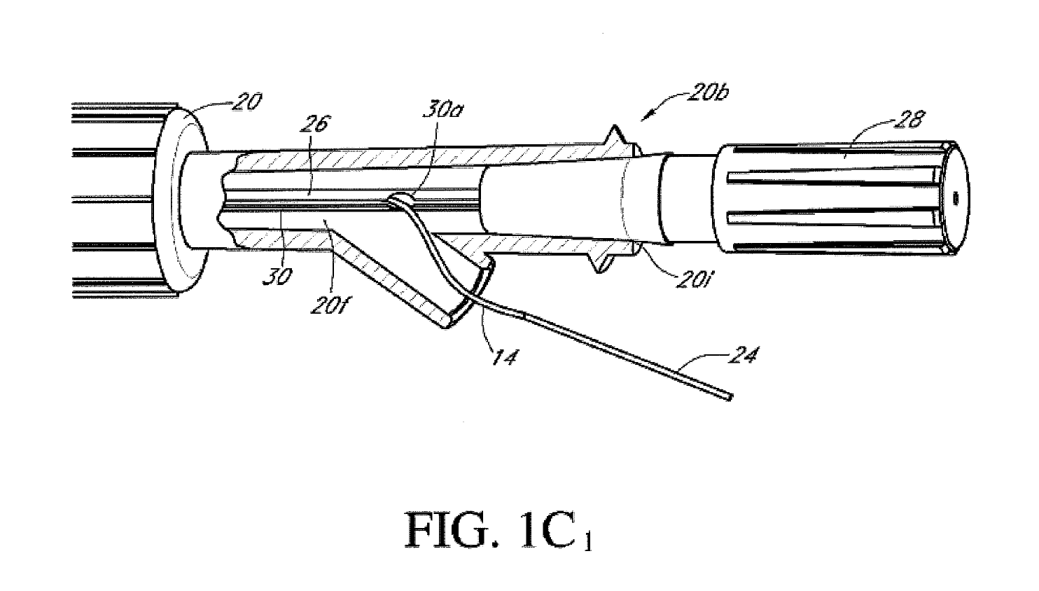

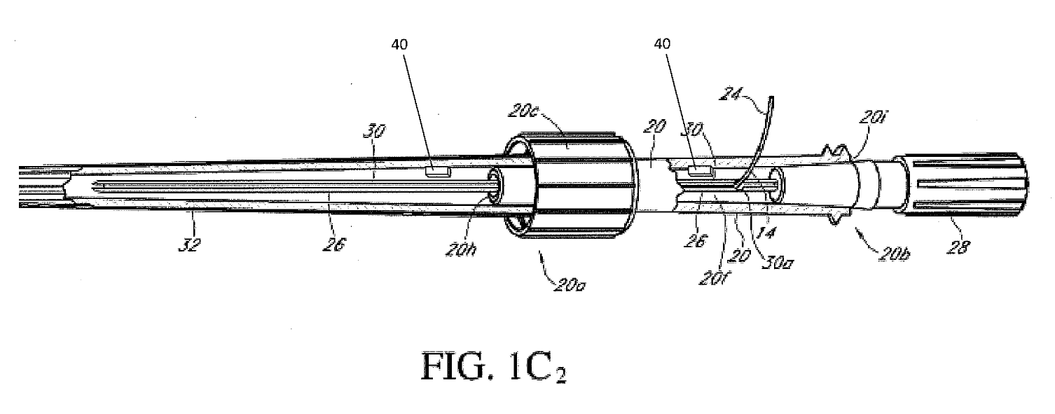

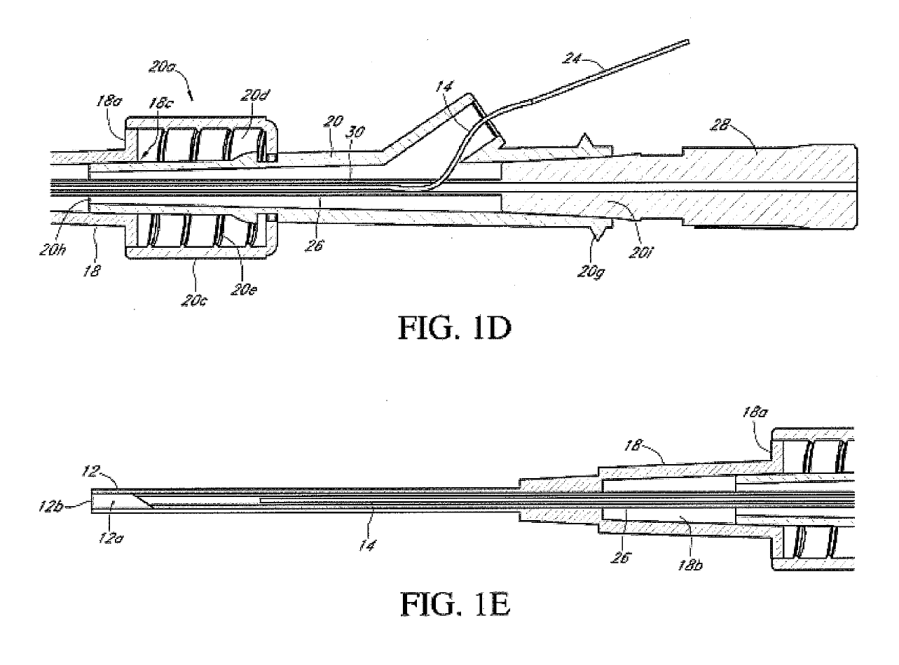

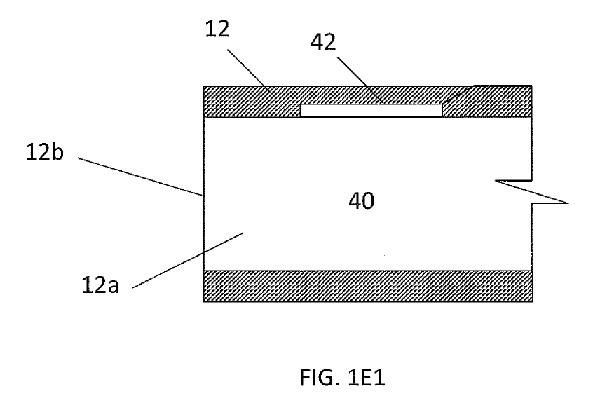

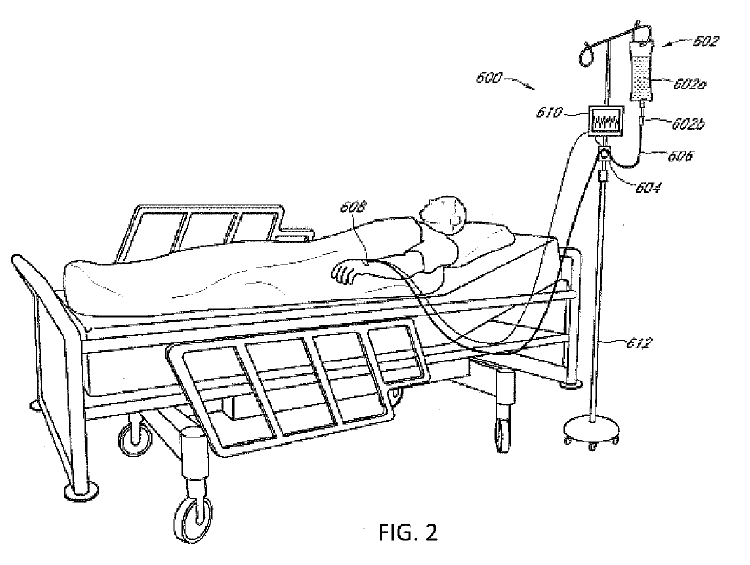

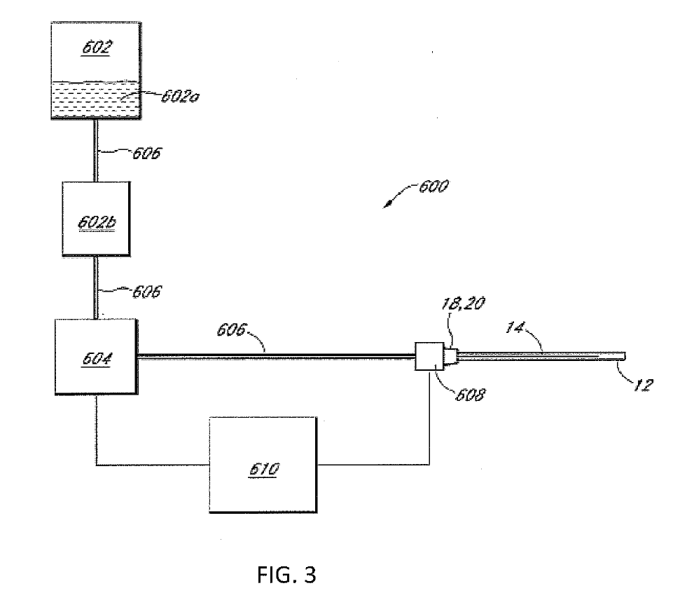

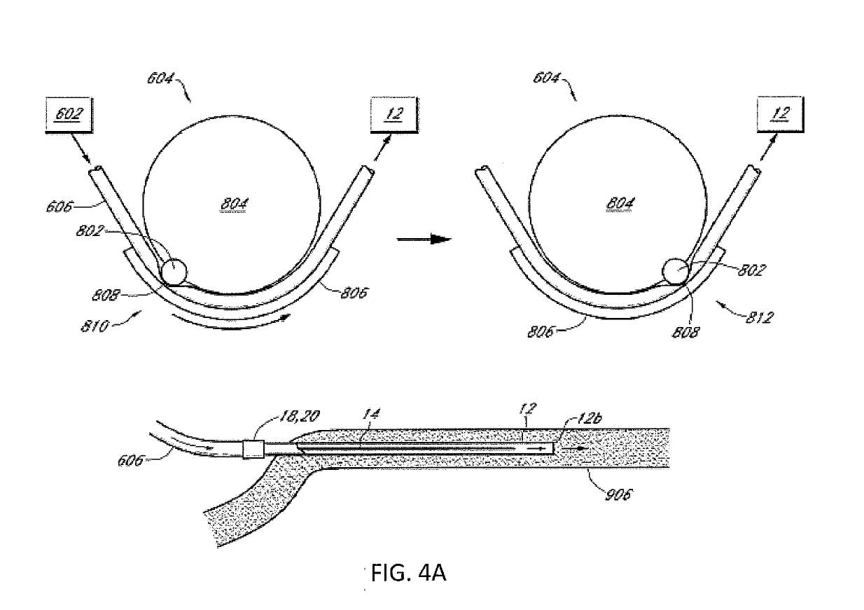

FIGS. 2 and 3 illustrate an integrated sensor system 600 (e.g., for use at the bedside) of one embodiment of the present invention which includes an analyte sensor 14 (e.g., a glucose sensor) and a vascular access device 12 (e.g., a catheter placed in a peripheral vein or artery) described below (see FIGS. 1A-1E), and which includes at least one fluid reservoir 602 (e.g., a bag of calibration or IV hydration solution), a flow control device 604 (e.g., to control delivery of an infusion fluid 602a from the reservoir to the host via the catheter), a local analyzer 608 and a remote analyzer 610. In some embodiments, the analyte sensor is configured to reside within the catheter lumen 12a (see FIGS. 1A-1E). In some embodiments, the sensor is disposed within the catheter such the sensor does not protrude from the catheter orifice 12b. In other embodiments, the sensor is disposed within the catheter such that at least a portion of the sensor protrudes from the catheter orifice. The analyte sensor and vascular access device used in the integrated sensor system 600 can be any types known in the art, such as but not limited to analyte sensors and vascular access devices described herein. For convenience, the vascular access device 12 will be referred to as a catheter herein. However, one skilled in the art appreciates that other vascular access devices can be used in place of a catheter.

FIGS. 1A to 1E illustrate one embodiment of an exemplary analyte sensor system 10 for measuring an analyte (e.g., glucose, urea, potassium, pH, proteins, etc.), that can be used in the integrated sensor system 600. The analyte sensor system includes a catheter 12 configured to be inserted or pre-inserted into a host's blood stream. In clinical settings, catheters are often inserted into hosts to allow direct access to the circulatory system without frequent needle insertion (e.g., venipuncture). Suitable catheters can be sized as is known and appreciated by one skilled in the art, such as but not limited to from about 1 French (0.33 mm) or less to about 30 French (10 mm) or more; and can be, for example, 2-20 French (3 French is equivalent to about 1 mm) and/or from about 33 gauge or less to about 16 gauge. Additionally, the catheter can be shorter or longer, for example 0.75, 1.0, 1.25, 1.5, 1.75, 2.0 inches in length or longer. The catheter can be manufactured of any medical grade material, such as but not limited to polymers and glass as described herein. A catheter can include a single lumen or multiple lumens. A catheter can include one or more perforations, to allow the passage of host fluid through the lumen of the catheter.

Referring now to FIGS. 1A to 1E, in some embodiments, the catheter 12 is a thin, flexible tube having a lumen 12a, such as is known in the art. In some embodiments, the catheter can be rigid; in other embodiments, the catheter can be custom manufactured to desired specifications (e.g., rigidity, dimensions, etc). The catheter can be a single-lumen catheter or a multi-lumen catheter. At the catheter's proximal end is a small orifice 12b for fluid connection of the catheter to the blood stream. At the catheter's distal end is a connector 18, such as a leur connector or other fluid connector known in the art.

The illustrations of FIGS. 1A to 1E show one exemplary embodiment of the connector 18 including a flange 18a and a duct 18b. In the exemplary embodiment, the flange 18a is configured to enable connection of the catheter to other medical equipment (e.g., saline bag, pressure transducer, blood chemistry device, and the like) or capping (e.g., with a bung and the like). Although one exemplary connector is shown, one skilled in the art appreciates a variety of standard or custom made connectors suitable for use with the preferred embodiments. The duct 18b is in fluid communication with the catheter lumen and terminates in a connector orifice 18c.

The catheter is inserted into the host's blood stream, such as into a vein or artery. Generally, prior to and during insertion, the catheter is supported by a hollow needle or trochar (not shown). For example, the supported catheter can be inserted into a peripheral vein or artery, such as in the host's arm, leg, hand, or foot. Typically, the supporting needle is removed (e.g., pulled out of the connector) and the catheter is connected (e.g., via the connector 18) to IV tubing and a saline drip, for example. However, the catheter can also be configured to operatively couple to medical equipment, such as a sensor system of one embodiment of the present invention.

The catheter and the analyte sensor are configured to indwell within the host's blood stream in vivo. An indwelling medical device, such as a catheter or implant, is disposed within a portion of the body for a period of time, from a few minutes or hours to a few days, months, or even years. An indwelling catheter is typically inserted within a host's vein or artery for a period of time, often 2 or more days, a month, or even a few months. Use of an indwelling catheter permits continuous access of an analyte sensor to a blood stream while simultaneously allowing continuous access to the host's blood stream for other purposes, for example, the administration of therapeutics (e.g., fluids, drugs, etc.), measurement of physiologic properties (e.g., blood pressure), fluid removal, and the like.

Referring again to FIGS. 1A to 1E, the system 10 also includes the analyte sensor 14 configured to extend through the catheter lumen 12a (see FIG. 1E), out of the catheter orifice 12b and into the host's blood stream by about 0.010 inches to about 1 inch, or shorter or longer lengths. In some embodiments, however, the sensor may not extend out of the catheter, for example, can reside just inside the catheter tip.

Various components of the electronics of the sensor system can be disposed on or proximal to the analyte sensor, such as but not limited to disposed on a fluid coupler 20 of the system, such as the embodiment shown in FIG. 1A. In another embodiment, wherein the sensor is integrally formed on the catheter, the electronics are disposed on or proximal to the connector 18. In some embodiments, only a portion of the electronics (e.g., the potentiostat) is disposed on the device (e.g., proximal to the sensor), while the remaining electronics are disposed remotely from the device, such as on a stand or by the bedside. In a further embodiment, a portion of the electronics can be disposed in a central location, such as a nurse's station.

The sensor 14 is configured to measure the concentration of an analyte within the host's blood stream. Preferably, the sensor includes at least one electrode, for example a working electrode; however any combination of working electrode(s), reference electrode(s), and/or counter electrode(s) can be implemented. Preferably, the sensor 14 includes at least one exposed electroactive area (e.g., working electrode), a membrane system (e.g., including an enzyme), a reference electrode (proximal to or remote from the working electrode), and an insulator material. In some embodiments, the sensor is configured to measure glucose concentration.

Referring to FIGS. 1A to 1E, the sensor has a proximal end 14a and a distal end 14b. At its distal end 14b, the sensor 14 is associated with (e.g., connected to, held by, extends through, and the like) a fluid coupler 20 having first and second sides (20a and 20b, respectively). The fluid coupler is configured to mate (via its first side 20a) to the catheter connector 18. In one embodiment, a skirt 20c is located at the fluid coupler's first side and includes an interior surface 20d with threads 20e (see FIGS. 1D and 1E). In this embodiment, the fluid coupler is configured to mate with the connector flange 18a, which is screwed into the fluid coupler via the screw threads. However, in other embodiments, the fluid coupler may be configured to mate with the connector using other mating configuration, for example, a snap-fit, a press-fit, an interference-fit, and the like, and can include a locking mechanism to prevent separation of the connector and fluid coupler. The fluid coupler 20 includes a lumen 20f extending from a first orifice 20h on its first side 20a to a second orifice 20i located on the fluid coupler's second side 20b (FIGS. 1C1 to 1E). When the catheter connector is mated with the fluid coupler, the catheter's lumen 12a is in fluid communication with the fluid coupler's lumen 20f via orifices 18c and 20h.

In the exemplary embodiment, the second side 20b of the fluid coupler 20 is configured to be operably connected to IV equipment, another medical device or to be capped, and can use any known mating configuration, for example, a snap-fit, a press-fit, an interference-fit, and the like. In one exemplary embodiment, the second side 20b is configured to mate with a saline drip, for delivery of saline to the host. For example, the saline flows from an elevated bag of sterile saline via tubing, through the fluid coupler, through the catheter and into the host's blood system (e.g., vein or artery). In another embodiment, a syringe can be mated to the fluid coupler, for example, to withdraw blood from the host, via the catheter.

Referring to the exemplary embodiment of FIGS. 1A and 1E, at least a portion of the sensor 14 passes through the fluid coupler 20 (e.g., the fluid coupler lumen 20f) and is operatively connected to sensor electronics via a hardwire 24. In alternative embodiments however, the sensor electronics can be disposed in part or in whole with the fluid coupler (e.g., integrally with or proximal to) or can be disposed in part or in whole remotely from the fluid coupler (e.g., on a stand or at the bed side).

Referring again to FIGS. 1A to 1E, a protective sheath 26 is configured to cover at least a portion of the sensor 14 during insertion, and includes hub 28 and slot 30. In general, the protective sheath protects and supports the sensor prior to and during insertion into the catheter 12 via the connector 18. The protective sheath includes a hub 28, for grasping the sheath (e.g., while maintaining sterilization of the sheath). In this embodiment, the hub additionally provides for mating with the second side 20b of the fluid coupler 20, prior to and during sensor insertion into the catheter. The slot of the protective sheath is configured to facilitate release of the sensor therefrom. After the sensor has been inserted into the catheter, the hub is grasped and pulled from the second side of the fluid coupler. This action peels the protective sheath from the sensor (e.g., the sensor slides through the slot as the sheath is removed), leaving the sensor within the catheter.

FIGS. 1C1 and 1C2 are cross-sectional views (not to scale) of the fluid coupler, including a protective sheath 26, a sensor 14, and a cap 32 (cap to be removed prior to insertion) in one embodiment. The protective sheath 26 extends through the fluid coupler and houses the sensor, for sensor insertion into a catheter. The protective sheath includes an optional outlet hole 30a, through which the sensor extends and a slot 30 along a length of the protective sheath that communicates with the outlet hole and enables the protective sheath to be removed after the sensor has been inserted into the host's body. The protective sheath includes a hub 28 for ease of handling.

In practice, prior to insertion, the cap 32 over the protective sheath is removed as the health care professional holds the glucose sensor by the fluid coupler 20. The protective sheath 26, which is generally more rigid than the sensor but more flexible than a needle, is then threaded through the catheter so as to extend beyond the catheter into the blood flow (e.g., by about 0.010 inches to about 1 inches). The protective sheath is then removed by sliding the sensor through the (optional) outlet hole 30a and slotted portion 30 of the sheath (e.g., by withdrawing the protective sheath by pulling the hub 28). Thus the sensor remains within the catheter; and the fluid coupler 20, which holds the sensor 14, is coupled to the catheter itself (via its connector 18). Other medical devices can be coupled to the second side of the fluid coupler as desired. The sensor electronics (e.g., adjacent to the fluid coupler or otherwise coupled to the fluid coupler) are then operatively connected (e.g., wired or wirelessly) to the sensor for proper sensor function as is known in the art.

In some embodiments, as shown for example in FIG. 2, at least one electronics module is included in the local and/or remote analyzers 608, 610 respectively, for controlling execution of various system functions, such as but not limited to system initiation, sensor calibration, movement of the flow control device 604 from one position to another, collecting and/or analyzing data, and the like. In preferred embodiments, the components and functions of the electronics module can be divided into two or more parts, such as between the local analyzer 608 and remote analyzer 610. The flow control device 604 may also be considered to include functions of the local and remote analyzers 608, 610.

Various components of the electronics of the sensor system can be disposed on or proximal to the analyte sensor, such as, but not limited to, being disposed on the fluid coupler 20 shown in FIG. 1A. In another embodiment, wherein the sensor is integrally formed on the catheter and the electronics are disposed on or proximal to the connector 218. In some embodiments, only a portion of the electronics (e.g., the potentiostat) is disposed on the device (e.g., proximal to the sensor), while the remaining electronics are disposed remotely from the device, such as on a stand or by the bedside. In a further embodiment, a portion of the electronics can be disposed in a central location, such as a nurse's station.

The electronics associated with the analyte sensor 14 and/or the local analyzer 608 and/or the remote analyzer 610 include a processor module that includes the central control unit that controls the processing of the integrated sensor system 600. In some embodiments, the processor module includes a microprocessor, however a computer system other than a microprocessor can be used to process data as described herein, for example an application-specific integrated circuit (ASIC) can be used for some or all of the sensor's central processing. The processor typically provides semi-permanent storage of data, for example, storing data such as sensor identifier (ID) and programming to process data streams (for example, programming for data smoothing and/or replacement of signal artifacts). The processor additionally can be used for the system's cache memory, for example for temporarily storing recent sensor data. In some embodiments, the processor module comprises memory storage components such as ROM, RAM, dynamic-RAM, static-RAM, non-static RAM, EEPROM, rewritable ROMs, flash memory, and the like.

Referring to FIGS. 2 and 3, in preferred embodiments, the integrated sensor system 600 includes at least one reservoir 602 that contains an infusion fluid 602a, such as but not limited to reference (e.g., calibration), hydration and/or flushing solutions. For simplicity, the infusion fluid 602a will be referred to herein as a solution 602a. Although a wide variety of infusion fluids may be used with the present invention, there is a particular need for having a solution without an anticoagulant component, e.g., a solution without citrate, heparin or other chemicals that may affect function of the patient's vascular and blood tissues in undesired ways.

The reservoir 602 includes a container such as but not limited to an IV bag. In other embodiments, the reservoir 602 can include two or more IV bags, or any other sterile infusion fluid container. In some embodiments, the reservoir 602 is a multi-compartment container, such as but not limited to a multi-compartment IV bag. If two or more solutions 602a (e.g., calibration solutions, flush solutions, medication delivery solutions, etc.) are used, the solutions 602a can be contained in two or more IV bags or in a multi-compartment IV bag, for example. In some embodiments, it is preferred to use a single solution 602a. Use of a single solution 602a for calibration, catheter flushing and the like simplifies the system 600 by reducing the complexity and/or number of system 600 components required for system 600 function.

In one embodiment, the solution 602a is an analyte-containing solution that can be used as a reference or standard for sensor 14 calibration (generally referred to as a calibration solution in the art). For example, the solution 602a may contain dextrose or glucose at a concentration of from about 0 mg/dl to about 400 mg/dl. Preferably, the solution 602a is also a flushing solution with solvent properties to facilitate freeing of a sample, such as blood and associated clots, off the sensor 14 and out of the catheter 12. For example, the solution 602a may be an isotonic saline solution. Less preferred, because one desired advantage of the present invention is to ameliorate the use of anticoagulant components, the solution 602a may contain a sufficient concentration of an anticoagulant to facilitate the mechanical flushing of blood clots in and/or near the catheter 14. For example, a citrate concentration equal to or less than about 2 wt % may be used in the solution. The solution 602a may also contain a sufficient concentration of or antimicrobial to substantially prevent infection in and/or near the catheter. In one exemplary embodiment, the reservoir 602 is a 500 ml bag containing a sterile solution 602a including 0.9% sodium chloride in water (e.g., normal saline) and 100 mg/dl dextrose.

The solution 602a can be provided to the user in a variety of ways, depending upon local hospital protocol and/or physician preference. In some embodiments, the solution 602a is supplied pre-mixed (e.g., an IV bag containing sodium chloride and dextrose), such that fluid reservoir 602 can be connected to an infusion set and infused into the host with minimal effort. In other embodiments, one or more of the solution components 602a can be provided separately, such that the final solution 602a is prepared at the host's bedside, at the nurse's station or in the hospital pharmacy, for example.

In various preferred embodiments, the solutions 602a are administered with standard IV administration lines, such as those commonly used today, such as a sterile, single-use IV set, referred to herein as tubing 606.

As shown in FIG. 2, the reservoir 602 is held by a support 612. The support 612 can take many forms, such as an elevated support. In some embodiments, the support 612 is an IV pole, such those commonly used in medical care facilities. In some embodiments, the reservoir 602 is suspended on the support 612, and the height of the reservoir 602 can be adjusted (e.g., raised or lowered) to modulate solution 602a discharge from the reservoir 602.

The flow control device 604 of one embodiment of the present invention includes one or more valves and is configured to control fluid delivery to the host and sample take-up (e.g., drawing blood back into the catheter until at least the sensor's electroactive surfaces are contacted by the blood). In some embodiments, wherein an internal calibration is performed, an infusion fluid (e.g., calibration solution 602a) flows over the indwelling sensor 14 and is infused into the host. For calibration, analyte in the solution 602a can be measured when the sensor 14's electroactive surfaces are in contact with the solution 602a. After calibration, the system is configured such that a sample (e.g., blood or other bodily fluid) is drawn into contact the sensor's electroactive surfaces (e.g., by drawing blood back into the catheter). When the sample contacts the electroactive surfaces, the sample's analyte concentration can be detected by the sensor 14. After the sample's analyte concentration is detected, it can then be returned to the host.

Preferably, to facilitate continuous sampling, the integrated sensor system 600 cycles between calibration (e.g., measurement of a reference calibration solution), flushing (as described in more detail hereinbelow) and measurement (e.g., of a sample, such as blood, glucose concentration). The system 600 continues operation in this cyclical manner, until the system 600 is either disconnected from the host or turned off for a period of time (e.g., during movement of the host from one location to another) or some special flushing condition arises and is detected, as described hereinbelow. For example, in one embodiment, the system 600 cycles between the calibration, flushing and measurement steps from about every 30 seconds or less to about every 2 hours or more. Less than about 5, 7 or 10 minutes, however, corresponds more closely to the expected Nyquist frequency of glucose variations in a patient's blood. In some embodiments, the cycle time is dynamic and dependent upon sensors that detect a reference solution (e.g., calibration solution) and/or sample (e.g., blood) at the electroactive surfaces and/or clotting in the system.

As described below, in certain circumstances and/or other embodiments, the system 600 can perform additional steps, such as, but not limited to, an adapted flushing step, a keep vein open step (KVO), or an adapted infusion step, and the like.

Still referring to FIGS. 2 and 3, in some embodiments, a flow regulator 602b controls the solution 602a flow rate from the reservoir 602 to the flow control device 604, which is described below. A variety of flow regulators can be used with the preferred embodiments, including but not limited to pinch valves, such as rotating pinch valves and linear pinch valves, cams and the like. In one exemplary embodiment, the flow regulator 602b is a pinch valve, supplied with the IV set and located on the tubing 606 adjacent to and below the drip chamber. In some embodiments, a flow regulator 602b controls the flow rate from the reservoir 602 to a flow control device 60. In some embodiments, a flow regulator is optional; and a flow control device 604 controls the flow rate (e.g., from the reservoir 602 to the catheter 14, described elsewhere herein).

In preferred embodiments, the integrated sensor system 600 includes a flow control device 604. The flow control device 604 is configured to regulate the exposure of the sensor 14 to the solution 602a and to the host sample (e.g., blood or other bodily fluid). The flow control device 604 can include several electromechanical devices for regulating flow, such as but not limited to valves, cams, pumps, and the like. In one exemplary embodiment, the flow control device 604 includes a simple linear pinch valve. In another exemplary embodiment, the flow control device 604 includes two or more linear pinch valves. In still another exemplary embodiment, the flow control device 604 includes a pump, such as volumetric infusion pumps, peristaltic pumps, piston pumps and syringe pumps. In still other exemplary embodiments, the flow control device 604 can be configured to vary the pressure at the reservoir 602, such as but not limited to a pressure cuff around an IV bag and/or raising/lowering the reservoir adjust head pressure.

In some embodiments, sample is taken up into the same catheter lumen 12a through which the solution 602a is infused into the host (described elsewhere herein). Thus, it is preferred that mixing of the sample and the solution 602a is prevented. Similarly, it can be advantageous to detect when the sensor 14 is in contact with undiluted sample and/or undiluted solution. In some preferred embodiments of the integrated sensor system 600, the flow control device 604 is configured to substantially prevent mixing of two or more fluids, such as but not limited to the solution 602a and a host sample (e.g., blood). In preferred embodiments, mixing can be substantially prevented by a combination of factors, including specific gravity and flow rate. It is known that two solutions with different specific gravities tend not to mix, provided that the fluids are moved at a sufficiently slow rate (e.g., flow rate). Human whole blood has a specific gravity of about 1.05-1.06, while an infusion solution of 5% dextrose and 0.225% NaCl has a specific gravity of about 1.0189. Due to the difference in specific gravities, a blood sample and the solution 602a tend to resist mixing within the tubing 606 when the flow rate is sufficiently slow. In preferred embodiments, the sample and the solution 602a are moved within the catheter lumen 12a at a rate such that substantially no mixing occurs therebetween. In some embodiments, the flow rate is from about 0.001 ml/min or less to about 2.0 ml/min or more. In preferred embodiments, the flow rate is from about 0.01 ml/min to about 1.0 ml/min. In one exemplary preferred embodiment, the flow rate is from about 0.02 ml/min to about 0.35 ml/min. In another exemplary preferred embodiment, the flow rate is from about 0.0.02 ml/min to about 0.2 ml/min. In yet another exemplary preferred embodiment, the flow rate is from about 0.085 ml/min to about 0.2 ml/min.

The flow control device 604 can include a variety of fluid flow-regulating devices. In some embodiments, the flow control device 604 includes one or more valves, such as but not limited to linear and non-linear roller valves, linear and non-linear pinch valves, bi-directional valves (either linear or non-linear), peristaltic rollers, cams, combinations thereof, and the like. In some other embodiments, the flow control device 604 is configured to generate sufficient “head pressure” to overcome the host's blood pressure such that the solution 602a is infused into the host at a controlled rate; this can include elevating the fluid reservoir 602 (e.g., gravity fed) and using a valve to control the fluid flow rate out of the reservoir 602 and into the host.

In one exemplary embodiment, the flow control device 604 is a rotating pinch valve that has first and second positions. The valve can move between the two positions, for example, backward and forward, and thereby move fluids in and out of the catheter. Namely, solution 602a can be moved from the reservoir 602, over the electroactive surfaces of the sensor 14 and into the host; and the sample can be drawn up from the host, to cover the electroactive surfaces of the sensor 14, and then pushed back into the host, by movement of the valve between the first and second positions.

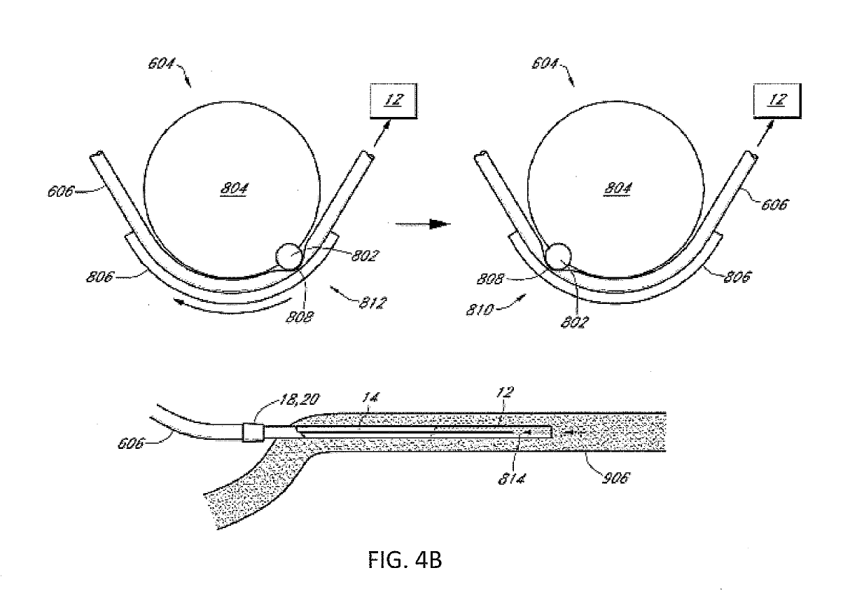

In another exemplary embodiment, the flow control device includes a rotating pinch valve as described with reference to FIGS. 4A through 4C. Although FIGS. 4A to 4C describe one implementation of a rotating pinch valve that can be implemented with the sensor system, some alternatives include rotating pinch valves with multiple pinch surfaces, for example around the circumference of the rotatable axle (FIG. 4, 804), which enables the use of one valve for multiple infusion fluids (e.g., using multiple IV lines).

In a further embodiment, the flow control device 604 is a computer controlled rolling pinch valve that acts on the exterior of sterile tubing 606 in order to control the gravity flow of a solution 602a from an elevated fluid reservoir 602 into the host. The flow control device 604 is configured to pinch and roll a small volume of tubing 606 such that a sample of host blood is drawn up into the catheter 12 (e.g., with a sensor 14 disposed therein) for analyte measurement, and to then push the sample back into the host with a solution (e.g., the calibration solution 602a). In general, the flow control device 604 is configured to oscillate between drawing up a blood sample and allowing flow of the calibration solution 602a at a predetermined rate. In some embodiments, the flow control device 604 includes at least one “hard stop” that ensures that the flow control device 604 does not move to a position that could endanger and/or injure the host, such as by draining the IV bag 602 of fluid 602a or inappropriately (e.g., excessively) withdrawing blood, for example.

In one exemplary embodiment, the sensor 14 is configured to reside within the catheter lumen 12a (e.g., not protrude from the catheter tip); and the flow control device 604 is configured to draw back a sample into the catheter lumen 12a such that at least the sensor's electroactive surfaces are contacted by the sample. In some embodiments, the sensor 14 is configured such that its electroactive surfaces are at or adjacent to its tip, and the flow control device 604 is configured to take up sample into the catheter lumen 12a until the sample covers at least the electroactive surfaces. In some embodiments, the electroactive surfaces are distal from the sensor's tip and sample is drawn farther back into the catheter lumen 12a until the sample covers the electroactive surfaces. In some embodiments, the tip of the sensor is disposed about 3 cm, 2 cm, or 1 cm or less from a tip of the catheter.

In some embodiments, the sample taken up into the catheter's lumen 12a covers only a portion of the sensor's in vivo portion. In other embodiments, the sample taken up into the catheter's lumen 12a covers the entire in vivo portion of the sensor 14. In some embodiments, a sample volume of from about 1 μl or less to about 2 ml or more is taken up into the catheter 12 and is sufficient to cover at least the electroactive surfaces of the sensor 14. In some preferred embodiments, the sample volume is from about 10 μl to about 1 ml. In some preferred embodiments, the sample volume is from about 20 μl to about 500 μl. In other preferred embodiments, the sample volume is from about 25 μl to about 150 μl. In more preferred embodiments, the sample volume is from about 2 μl to about 15 μl.

In preferred embodiments, the sample taken up into the catheter's lumen 12a remains within the in vivo portion of the catheter 12. For example, in some embodiments, the sample is not drawn so far back into the catheter 12 that it enters the ex vivo portion of the catheter 12, the tubing 606 or the reservoir 602. In some embodiments, however, the sample can be drawn back as far as the catheter but not into the IV tubing. In some embodiments wherein the catheter 12 is implanted in a host, the blood sample never leaves the host's body (e.g., a plane defined by the host's skin). In some embodiments wherein the catheter 12 is implanted in an extracorporeal device, the sample does not substantially exit the extracorporeal device. In preferred embodiments, wherein blood is taken up into the catheter 12, the blood is returned to the host (or extracorporeal device), which is described elsewhere herein. In preferred embodiments, the sample is blood taken up from the host's circulatory system and into the catheter 12 disposed within the circulatory system.

In some embodiments, the remote analyzer 610 controls the function of the flow control device 604. In some embodiments, the flow control device includes electronics configured to control the flow control device. The flow control device 604 can be configured to perform a number of steps of operation, which are discussed below. Depending upon the system configuration and physician preferences, in some embodiments, one or more of the steps can be performed. In some embodiments, all of the steps are performed. In some embodiments, the steps of operation can be performed in the order in which they are presented herein. In other embodiments, the order of steps of operation can be varied (e.g., repeated, omitted, rearranged), depending upon various parameters, such as but not limited to the calibration solution 602a selected, the particular infusion set selected, catheter 12 size, host condition, analyte of interest, type of sample and location of sample collection, integration with third party devices, additional infusion of fluids and the like.

FIGS. 4A through 4C are schematic illustrations of a flow control device in one exemplary embodiment, including its relative movement/positions and the consequential effect on the flow of fluids through the sensor/catheter inserted in a host in a situation where no clotting or occlusion of the catheter catheter 12 is detected. In general, steps performed by the flow control device 604, include the steps of: contacting the sensor 14 with calibration solution 602a (including sensor calibration) and contacting the sensor with a biological sample to be measured. In some embodiments, additional steps can be taken, such as but not limited to keep a vein open (KVO) step and, a regular wash step or a flush step in special circumstances when clotting is detected. In the exemplary embodiment presented in FIGS. 4A though 4C, the flow control device 604 is a roller valve configured to move between at least two positions, 810 and 812, respectively. Movement of the flow control device 604 between positions 810 and 812 effectively concurrently moves the pinch point 808 (e.g., the point at which tubing 606 is pinched) between positions 810 and 812. Additional flow control device positions are discussed below.

The top of FIGS. 4A through 4C are schematic drawings illustrating positions of the flow control device 604. The bottom of FIGS. 4A through 4C, are a cut-away views of an implanted catheter 12, including an indwelling sensor 14, illustrating the corresponding activity at the implantation site, in response to movements of the flow control device 604. For simplicity, for purposes of discussion only, it is assumed that the catheter 12 is implanted in a host's vein and that the sensor 12 does not protrude from the catheter's orifice 12b. However, the catheter 14 could be implanted into any vessel of the host or into a variety of extracorporeal devices discussed elsewhere herein.

Step One: Contacting Sensor with Calibration Solution

In general, the system is configured to allow a calibration solution to contact the sensor using a flow control device such as a pump, valve or the like. In some embodiments, such as shown in FIGS. 4A through 4C, the flow control device 604 is a valve configured with a first structure 802 and a second structure 806. For convenience, the first structure 802 is depicted as a roller connected to a rotatable axle 804, other flow control devices can be configured to utilize the concepts and/or functions described herein. In general, when the flow control device is a valve, the valve is configured to allow no flow, free flow and/or metered flow through movement of the valve between one or more discreet positions.

In the embodiment shown in FIGS. 4A through 4C, the flow control device 604 is configured such that a tube 606 threaded between the first and second structures 802, 806 (e.g., between the roller and the surface against which the roller presses) is compressed substantially closed. For convenience, the compressed location on the tubing is referred to herein as the “pinch point” 808. In some embodiments, the flow control device 604 is configured such that the pinch point is moved along the tubing, either closer to or farther from the host. As the pinch point 808 is moved closer to the host, the tube 606 is progressively compressed, causing fluid (e.g., solution 602) to be pushed into the host's vascular system (see the corresponding illustration of the sensor within the host's vessel at the bottom of FIG. 4A), at the catheter 12 implantation site. Conversely, as the pinch point 808 is moved away from the host, the portion of tubing 606 on the host side of the pinch point 808 progressively expands, causing sample (e.g., blood) to be drawn up into the catheter lumen 12a.

In the exemplary embodiment shown in FIG. 4A (bottom), the catheter 12 is implanted in the host's vein 906 (or artery). A sensor 14 is disposed within the catheter 12. The catheter 12 is fluidly connected to a first end of tubing 606 that delivers the solution 602a to the catheter 12. The solution 602a can move out of the catheter 12 and a sample of blood 814 can move in and out of the catheter 12, via the catheter's orifice 12b.

Referring now to a calibration phase to be performed by the exemplary valve of FIG. 4A, in preferred embodiments, the flow control device 604 is configured to perform a step of contacting the sensor 14 with solution 602a, wherein the flow control device 604 moves from position 810 to position 812 (e.g., forward, toward the host/catheter). When the flow control device 604 moves from position 810 to position 812, the pinch point 808 is moved from position 810 to position 812. As the pinch point 808 is moved from position 810 to position 812, a first volume of the calibration solution 602a is pushed through the tubing 606, toward the catheter 12.

Referring again to the bottom of FIG. 4A, a second Volume of the solution 602a, which is substantially equal to the first volume, is pushed into the host's vein 906, in response to the first volume of solution 602a moving toward the host. As the second volume of solution 602a is pushed through the catheter 12 and into the host's vein the second volume contacts (e.g., bathes) the analyte sensor 14, including the analyte sensor's electroactive surfaces. In some embodiments, the volume (e.g., the first and second volumes of fluid) moved is from about 3 μl or less to about 1 ml or more. In some preferred embodiments, the volume is from about 10 μl to about 500 μl, or more preferably from about 15 μl to about 50 μl.

In general, the volume of fluid pushed through the catheter in a particular phase (e.g., calibration phase) is dependent upon the timing of the phase. For example, if a long phase, such as a 20 minute calibration phase (e.g., as compared to a shorter 5 minute phase) were selected, the volume of fluid pushed during the long phase would be 4× greater than the volume of fluid pushed during the shorter phase. Accordingly, one skilled in the art appreciates that the above described ranges of fluids infusion can be increased and/or decreased simply be increasing or decreasing the measurement phase and/or intervals (i.e., timing).

In preferred embodiments, the fluid is moved at a flow rate that is sufficiently slow that the calibration solution's temperature substantially equilibrates with the temperature of the tissue surrounding the in vivo portion of the catheter and/or temperature of bodily fluid (e.g., blood). In preferred embodiments, the flow rate is from about 0.25 μl/min or less to about 10.0 ml/min or more. In one exemplary embodiment, the flow control device 604 maintains a flow rate from about 0.5 μl/min or less to about 1.5 ml/min or more. In one preferred exemplary embodiment, the flow rate is from about 1 μl/min to about 1.0 ml/min. In one exemplary preferred embodiment, the flow rate is from about 0.01 ml/min to about 0.2 ml/min. In another exemplary preferred embodiment, the flow rate is from about 0.05 ml/min to about 0.1 ml/min. Notably, all of these flow rates are for normal operation where clots are not detected and some type of modification imposed in response thereto by the flow control device.

In some embodiments, the system is configured such that the speed of the movement between the first and second discreet positions is regulated or metered to control the flow rate of the fluid through the catheter. In some embodiments, the system is configured such that the time of movement between the first and second discreet positions is from about 0.25 to 30 seconds, preferably from about 0.5 to 10 seconds. In some embodiments, the system is configured such that an amount of pinch of the tubing regulates the flow rate of the fluid through the catheter. In some embodiments, the fluid flow is regulated through a combination of metering and/or pinching techniques, for example.

Preferably, the sensor is configured to measure a signal associated with the solution (e.g., analyte concentration) during the movement of the flow control device from position 810 to position 812 and/or during contact of the sensor 14 with the solution 602a. Electronics, such as an electronic module included in either the local or remote analyzer 608, 610 controls signal measurement and processing.

In general, a calibration measurement can be taken at any time during the flow control device 604 movement from position 810 to position 812, and including a stationary (stagnant) time thereafter. In some embodiments, one or more calibration measurements are taken at the beginning of the flow control device 604 movement from position 810 to position 812. In other embodiments, one or more calibration measurements are taken at some time in the middle of the flow control device 604 movement from position 810 to position 812. In some embodiments, one or more calibration measurements are taken near the completion of the flow control device 604 movement from position 810 to position 812. In some embodiments, one or more calibration measurements are taken after completion of the flow control device 604 movement from position 810 to position 812. In still other embodiments, the flow control device is positioned such that fluid can flow followed by positioning the flow control device such that there is no fluid flow (e.g., 0 ml/min) during the calibration measurement. In preferred embodiments, one or more calibration measurements are taken when the temperature of the solution 602a has substantially equilibrated with the temperature of the tissue surrounding the in vivo portion of the implanted catheter 12. Processing of calibration measurements and sensor calibration are described elsewhere herein.

Step Two: Sample Collection and Measurement

In general, the system is configured to allow a sample (e.g., blood) to contact the sensor using the flow control device. Referring now to the top of FIG. 4B, the flow control device 604 is configured to draw back (or take-in) a sample (e.g., blood) from the host. For example, to collect a sample, the flow control device 604 reverses and moves backward (e.g., away from the host/catheter), from position 812 to position 810, thereby causing the pinch point 808 to move away from the host. As the pinch point is moved from position 812 to position 810, the tube 606 (on the host side of the pinch point 808) expands (e.g., the tube volume increases).

Referring now to the bottom of FIG. 4B, as the tube volume increases, a small, temporary vacuum is created, causing sample 814 (e.g., blood) to be taken up into the catheter lumen 12a. In some embodiments, the flow control device 604 is configured to take up a sufficient volume of sample 814 such that at least the sensor's electroactive surfaces are contacted by the sample 814. In some embodiments, a sample volume of from about 1 μl or less to about 2 ml or more is taken up into the catheter 12 and is sufficient to cover at least the electroactive surfaces of the sensor 14. In some preferred embodiments, the sample volume is from about 10 μl to about 1 ml. In some preferred embodiments, the sample volume is from about 20 μl to about 500 μl. In other preferred embodiments, the sample volume is from about 25 μl to about 150 μl. In more preferred embodiments, the sample volume is from about 2 μl to about 15 μl.

In some embodiments, the rate of sample take-up is sufficiently slow that the temperature of the sample substantially equilibrates with the temperature of the surrounding tissue. Additionally, in some embodiments, the rate of sample take-up is sufficiently slow such that substantially no mixing of the sample 814 and solution 602a occurs. In some embodiments, the flow rate is from about 0.001 ml/min or less to about 2.0 ml/min or more. In preferred embodiments, the flow rate is from about 0.01 ml/min to about 1.0 ml/min. In one exemplary preferred embodiment, the flow rate is from about 0.02 ml/min to about 0.35 ml/min. In another exemplary preferred embodiment, the flow rate is from about 0.0.02 ml/min to about 0.2 ml/min. In yet another exemplary preferred embodiment, the flow rate is from about 0.085 ml/min to about 0.2 ml/min.

As described above, in some embodiments, the system is configured such that the speed of the movement between the first and second discreet positions is regulated or metered to control the flow rate of the fluid through the catheter. In some embodiments, the system is configured such that the time of movement between the first and second discreet positions is from about 0.25 to 30 seconds, preferably from about 0.5 to 10 seconds. In some embodiments, the system is configured such that the time of movement between the first and second discreet positions is from about 0.25 to 30 seconds, preferably from about 0.5 to 10 seconds. In some embodiments, the system is configured such that an amount of pinch of the tubing regulates the flow rate of the fluid through the catheter. In some embodiments, the fluid flow is regulated through a combination of metering and/or pinching techniques, for example.

Measurements of sample analyte concentration can be taken while the electroactive surfaces are in contact with the sample 814. An electronics module included in the local and/or remote analyzer 608, 610 controls sample analyte measurement, as described elsewhere herein. In some embodiments, one sample measurement is taken. In some embodiments, a plurality of sample measurements are taken, such as from about 2 to about 50 or more measurements and/or at a sample rate of between about 1 measurement per second and about 1 measurement per minute. In some embodiments, the rate is from about 1 measurement per 2 seconds to about 1 measurement per 30 seconds. In preferred embodiments, sample measurements are taken substantially continuously, such as but not limited to substantially intermittently, as described elsewhere herein.

Optional Step: Flush

In some exemplary embodiments, the flow control device 604 is configured to perform one or more steps, in addition to steps one and two, described above. A flush step, during which the sensor 14 and/or catheter 12 are substantially washed and/or cleaned of host sample, is one such optional step.

Referring now to the top of FIG. 4C, the exemplary flow control device 604 performs a flush step by moving forward from position 810 (e.g., toward the host/catheter), past position 812 (e.g., around and over the top of structure 804) and back to position 810. For convenience, the movement illustrated by an arrow in the top of FIG. 4C is referred to herein as the “flush movement.”

Referring now to the bottom of FIG. 4C, the flush movement pushes forward a volume of solution 602a (e.g., a third volume) that pushes the collected blood sample 814 into the host. In some embodiments, the third volume of solution 602a is substantially equal to the first and second volumes described above. In some embodiments, the flush movement is repeated at least one time. In some embodiments, the flush movement is repeated two, three or more times. With the exception of the first flush movement, which pushes the sample 814 back into the host, each repeat of the flush movement pushes a volume of solution 602a into the host, for example. In some embodiments, the flush movement pushes the third volume of solution 602a into the host at a rate of from about 0.25 μl/min or less to about 10.0 ml/min or more. In preferred embodiments the flush movement pushes the third volume of solution into the host at a rate of from about 1.0 μl/min to about 1.0 ml/min. In alternative embodiments, the flow control device 604 is moved to a fully opened position (e.g., no pinch) and the flow regulator 602b is set at a setting that allows more solution (e.g., an increased volume and/or at a faster rate) to infuse into the host than during the calibration phase (e.g., step one, above). In preferred embodiments, the flush movement washes enough blood off of the analyte sensor's electroactive surfaces that the sensor 14 can measure the solution 602a substantially without any interference by any remaining blood. In some embodiments, the flush step is incorporated into step one, above.

Generally, the solution 602a is flushed through the catheter 12, to ensure that a sufficient amount of the sample has been removed from the sensor 14 and the catheter lumen 12a, such that a calibration measurement can be taken. However, in some embodiments, sample is collected, measured and flushed out, followed by collection of the next sample, substantially without sensor calibration; the flush step can be executed between samples to ensure that the sample being analyzed is substantially uncontaminated by the previous sample. In some embodiments, a relatively extended flush is used, while in other embodiments the flush is just long enough to ensure no blood remains.

In some embodiments, the effectiveness of the flushing movement is dependent upon the solution 602a composition (e.g., concentrations of sodium chloride, glucose/dextrose, anticoagulant, etc.). Accordingly, the amount of solution 602a required to ensure that substantially no sample remains in the catheter 12 and/or on the sensor 14 can depend on the solution 602a composition. For example, relatively more flush movements may be required to completely remove all of the sample when a non-heparinized solutions is selected than when a heparinized solution is selected. In some embodiments, the effectiveness of the flushing movement is also dependent upon the flush flow rate. For example, a relatively faster flow rate can be more effective in removing sample from the sensor than a slower flow rate, while a slower flow rate can more effectively move a larger volume of fluid. Accordingly, in some embodiments, the number of flush movements selected is dependent upon the calibration solution and flow rate selected. In some embodiments, the flush step flow rate is from about 0.25 μl/min or less to about 10.0 ml/min or more, and last for from about 10 seconds or less to about 3 minutes or more. In one exemplary embodiment, about 0.33 ml of solution 602a is flushed at a rate of about 1.0 ml/min, which takes about 20 seconds.

In some embodiments, the flush step returns the sample 814 (e.g., blood) to the host, such that the host experiences substantially no net sample loss. Furthermore, the flush movement washes the sensor 14 and catheter lumen 12a of a sufficient amount of sample, such that an accurate calibration measurement (e.g.; of undiluted solution 602a) can be taken during the next step of integrated sensor system 600 operations. In some embodiments, the number of sequential flush movements is sufficient to only wash substantially the sample from the sensor 14 and catheter lumen 12a. In other embodiments, the number of sequential flush movements can be extended past the number of flush movements required to remove the sample from the sensor and catheter lumen, such as to provide additional fluid to the host, for example.

At the completion of the flush step, the flow control device 604 returns to step one, illustrated in FIG. 4A. In some embodiments, the steps illustrated in FIGS. 4A through 4C are repeated, until the system 600 is disconnected from the catheter/sensor, either temporarily (e.g., to move a host to an alternate location for a procedure) or permanently (e.g., at patient discharge or expiration of sensor life time). In some embodiments, additional optional steps can be performed.

Optional Step: Keep Vein Open (KVO)

Thrombosis and catheter occlusion are known problems encountered during use of an IV system, such as when the fluid flow is stopped for a period of time or flows at a too slow rate. For example, thrombi in, on and/or around the catheter 12, such as at the catheter's orifice 12b can cause an occlusion. Occlusion of the catheter can require insertion of a new catheter in another location. It is known that a slow flow of IV solution (e.g., saline or calibration fluid; with or without heparin) can prevent catheter occlusion due to thrombosis. This procedure is known as keep vein open (KVO).

In general, to infuse a fluid into a host, the infusion device must overcome the host's venous and/or arterial pressure. For example, during infusion of a hydration fluid, the IV bag is raised to a height such that the head pressure (from the IV bag) overcomes the venous pressure and the fluid flows into the host. If the head pressure is too low, some blood can flow out of the body and in to the tubing and/or bag. This sometimes occurs when the host stands up or raises his arm, which increases the venous pressure relative to the head pressure. This problem can be encountered with any fluid infusion device and can be overcome with a KVO procedure. KVO can maintain sufficient pressure to overcome the host's venous pressure and prevent “back flow” of blood into the tubing and/or reservoir.

In some embodiments, the flow control device 604 can be configured to perform a KVO step, wherein the fluid flow rate is reduced (but not completely stopped) relative to the calibration and/or wash flow rates. In preferred embodiments, the KVO flow rate is sufficient to prevent the catheter 12 from clotting off and is relatively lower than the flow rate used in step one (above). In preferred embodiments, the KVO flow rate is sufficient to overcome the host vessel pressure (e.g., venous pressure, arterial pressure) and is relatively lower than the flow rate used in step one (above).

In some embodiments, the KVO flow rate is from about 1.0 μl/min or less to about 1.0 ml/min or more. In some preferred embodiments, the KVO flow rate is from about 0.02 to about 0.2 ml/min. In some more preferred embodiments, the KVO flow rate is from about 0.05 ml/min to about 0.1 ml/min) In some embodiments, the KVO flow rate is less than about 60%, 50%, 40%, 30%, 20%, or 10% of the calibration and/or flush flow rate(s).

In some embodiments, the KVO step is performed for from about 0.25 minutes or less to about 20 minutes or more. In preferred embodiments, the solution 602a flows at a rate such that the temperature of the solution 602a substantially equilibrates with the temperature of the tissue surrounding the in vivo portion of the catheter 12. Advantageously, equilibrating the solution 602a temperature with that of the surrounding tissue reduces the effect of temperature on sensor 14 calibration and/or sample measurement, thereby improving sensor accuracy and consistency. In some embodiments, the KVO step can be incorporated into one or more of the flow control device steps of operation described elsewhere herein, including steps one and two, and the flush step, above.

The KVO step can be executed in one or more ways. In some embodiments, the flow control device 604 can be configured to move to at least one additional position, wherein the tube 606 is partially pinched. For example, the flow control device 604 is configured to move to a position such that the pinch point 808 is partially closed/open. For example, in the embodiment shown in FIGS. 4A through 4C, the flow control device 604 can be moved forward somewhat past position 812, such that the roller 802 causes the tube 606 to be partially pinched. In another example, the flow control device 604 can be moved backwards somewhat behind position 810, such that the roller 802 again causes the tube 606 to be partially pinched. In preferred embodiment, the amount of pinch can be adjusted such that the desired KVO flow rate can be achieved.

In some alternative embodiments, KVO is performed by moving the flow control device between positions 810 and 812 (e.g., see FIG. 4A) at a reduced speed, such that the flow rate is from about 0.1 μl/min or less to about 0.5 ml/min or more. In some embodiments, the system is configured such that the time of movement between the first and second discreet positions is from about 0.25 to 30 seconds, preferably from about 5 to 15 seconds. In some preferred embodiments, the tubing is pinched fully closed (e.g., between structures 802 and 806) during the movement from position 810 and 812 (e.g., see FIG. 4A). In some preferred embodiments, after the flow control device reaches position 812, the flow control device flips over the top and back to position 810 (e.g., see FIG. 4C) at a substantially rapid speed that the flow rate remains substantially unchanged. In an even further embodiment, during the KVO step the flow control device alternates between the slow and fast movements at least two times, such that the KVO step lasts a period of time.

As disclosed above, the flow control device 604 can be configured a variety of ways, which can require modifications to one or more of the steps of operation described above. For example, in some embodiments, the flow control device 604 can be configured to include a simple pinch valve, wherein the valve can be configured to open, close or partially open. In some embodiments, the flow control device 604 can be configured to include a non-linear rolling pinch valve, wherein the roller can move back and forth between opened, closed and partially opened positions, for example.

In some embodiments, the flow control device 604 can include one roller 802 (e.g., first structure) attached to an axle 804 and configured to press against a curved surface 806 (e.g., second structure), such that when the roller 802 is pressing against the curved surface 806 at or between positions 810 and 812, the tubing 606 is pinched completely closed and the flow control device 604 moves the roller 802 forward (e.g., toward the host). In one exemplary embodiment, the flow control device 604 can be configured to perform step one (above, contacting the sensor 14 with solution 602a) by moving the roller 802 forward (e.g., rotating from position 810 to 812, see FIG. 4A), thereby causing solution 602a to flow over the sensor 14. In some embodiments, the flow control device 604 is configured to perform step two (contacting the sensor 14 with sample) by moving the roller 802 backwards (e.g., rotating from position 812 to 810, see FIG. 4B), causing blood 814 to enter the catheter 12 and contact the sensor 14. Additionally, the flow control device 604 can be configured to perform a wash or KVO step by moving the roller 802 forward (from position 810) past position 812 and around the axle 804 until position 810 is again reached a plurality of times sequentially (e.g., see FIG. 4C). In a further example, the flow control device 604 includes two, three or more rollers 802 arranged about axle 804. In some embodiments, the flow control device includes a plurality of rollers arranged about the axle, wherein the flow control device performs KVO by rotating the rollers about the axle a plurality of times, to continuously push (e.g., for a period of time) the solution forward into the host.

In one alternative embodiment, back flow can be substantially stopped by incorporation of a one-way, pressure-controlled valve into the system, such as at or adjacent to the catheter or sensor connector, whereby fluid can flow into the host only when fluid pressure (e.g., head pressure) is applied to the reservoir-side of the valve. In other words, fluid can only flow in the direction of the host (e.g., toward the host), not backwards towards the reservoir. In some embodiments, the valve is a two-way valve configured such that the pressure required to open the valve is greater than the venous pressure, such that back flow is substantially prevented.

Dynamically Adaptable Draw and Flush

Although the above-described embodiments illustrating the optional flush and KVO steps are effective at clearing occlusions such as clotting or thrombosis, there is a need to optimize the fluid flow during the draw and/or flush steps to even further reduce occlusions. The integrated sensor system 600 in one embodiment employs the above-described analyte sensor 14, such as a three-wire (working, counter and blank electrodes) glucose sensor, with a membrane that is contained with the vascular access device 12, as shown in FIGS. 1A-1E. Use of heparin and other anticoagulant components in the fluid reservoir 602 are effective in that very low incidence of occlusion by clotting is shown when examined ex vivo. Heparin, however, has recently been resisted as an anticoagulant by hospitals around the world. Mitigation of clotting and other occlusions is more difficult using non-anticoagulant aided solutions.

Without being constrained to any particular theory, it is believed that observation of clots ex vivo is caused when the blood flushing phase is too short in duration and/or the blood draw into the vascular access device 12 is too long. In the former case, if more solution 602a is flowed across the sensor 14 while it is in the vascular access device 12, then the probability for clot formation would be reduced. Also, if the draw time into the vascular access device 12 is too long or too slow, the blood in the confined spaces of the vascular access device 12 could clot. Although a solution for this would be to simply increase flow of solution 602a during flush, decrease draw time and/or increase the rate of draw, these wholesale modifications represent a significant draw back by slowing the cycle of blood analyte measurement. To address these concerns, the present invention in one embodiment includes modifications to the system 600 to dynamically detect and respond to clotting and other occlusions only when needed and in a manner that minimizes lost cycle time. In other words, it is an objective of the following embodiments to optimize the flow rates and/or duration of the flush step and/or the draw step.

In an embodiment, the sensor system 600 includes one or more pressure sensors 40 disposed in the vascular access device or catheter 12, such as in the catheter lumen 12a, as shown in FIG. 1C2. These pressure sensors 40 dynamically sense pressure changes in the fluid column that starts at the distal end 12b of the catheter and extends up toward the fluid reservoir. The term pressure sensors as defined herein also includes flow sensors, wherein it is generally assumed that the response to an increase of pressure would be same as a decrease in flow or vice versa. Sensing of such pressure changes allows the sensor system 600 to respond with a modulation of the above-described flush/draw cycle to help clear clots and other occlusions.

The pressure sensor 40, for example, can be an ultra-miniature, solid-state pressure sensor used on mice manufactured by Scisense (London, Ontario, Canada) which can be mounted inside a catheter as small as 1.2 French. Advantageously, the Scisense pressure transducer has a recessed membrane and is customized for hemodynamic environments and has a smooth profile for minimizing interference with fluid flow.

The catheter 12 of the present invention could be modified to further accommodate the pressure sensor 40 by having a recess 42 cut within the internal passageway of the catheter that defines the lumen 12a, as shown by FIG. 1E1. As an alternative to an off-the-shelf pressure transducer, the transducer is built into the catheter by covering the recess 42 with a membrane with a stress/strain detector or piezoelectric component attached thereto to measure pressure variations. Also, although small size is an advantage, it should be noted that the present invention could be employed with a range of different sensing technologies. For example, there are optical pressure sensors that use fiber optics with a miniature pressure sensor on a distal end. As another alternative, a strain gauge could be affixed to a particularly flexible wall of a catheter 12 and correlated to pressure changes.

To ameliorate the size constraints, the pressure sensor 40 could be moved back up the fluid column toward the fluid reservoir 602 because the incompressibility of fluids generally results in immediate passage of pressure changes therethrough until blocked or dampened by intervening objects, such as the roller 802 or the compliance of tubing 606. For example, in another embodiment as shown in FIGS. 1C1 or 1C2, the pressure sensor 40 could be in the lumen 20f of the fluid coupler 20. An advantage in this instance is that the pressure sensor 40 could have a lead 44 that communicates through, or is passed along, hardwire 24, as shown in FIG. 1C1.

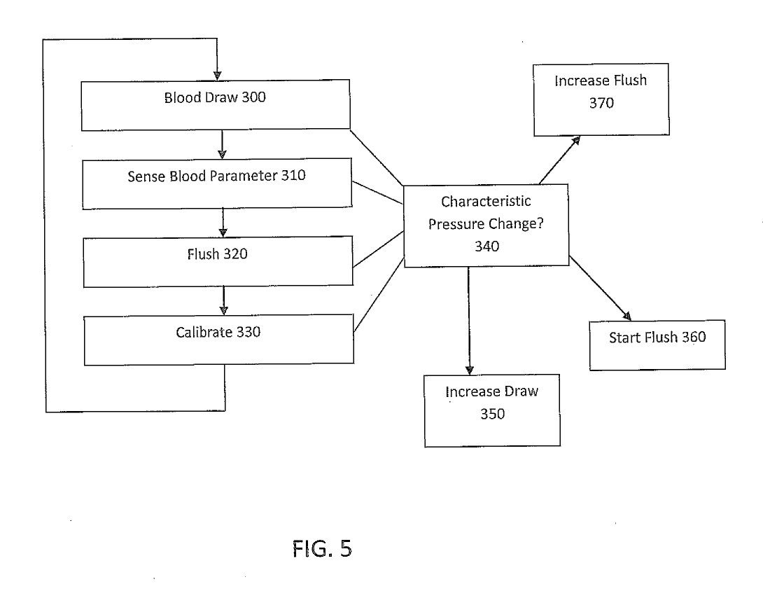

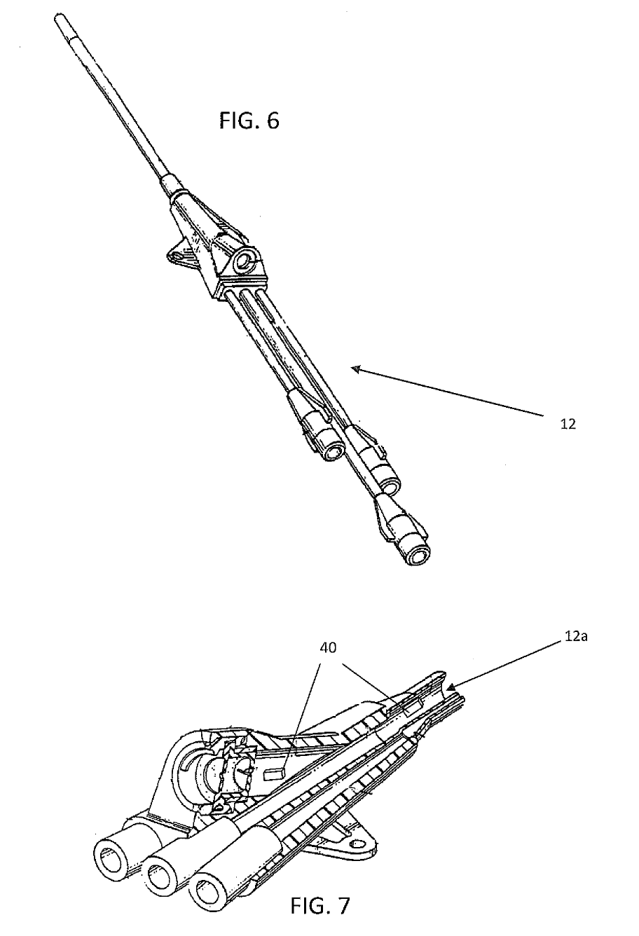

In yet another embodiment, the pressure sensor 40 could be mounted back in the hub or backform of a multi-lumen catheter 12 through which the analyte sensor 14 can be passed, as shown in FIGS. 6 and 7. This would provide further clearance for larger, more robust or cost-effective pressure sensors, such as the dynamic pressure transducer sold under the mark TRUWAVE (Edwards Lifesciences, Irvine, Calif.).

As yet another embodiment, multiple sensors could be employed along the fluid column extending back from the opening of the catheter 12, each with different sizes and advantageous characteristics to improve the accuracy, robustness and responsiveness of the pressure measurements. For example, if temperature effects are a concern an optical sensor could be combined with a more temperature sensitive piezoelectric sensor. It should be noted, however, that although a range of configurations and numbers of the pressure sensor 40 could be used, certain minimum thresholds are needed for response time and sensitivity to be preferred for the most effective fluid flow optimization.

The pressure sensor 40 (or sensors, as the case may be) is connected in communication through wires, leads and/or wirelessly to the remote or local analyzers 608, 610. As these analyzers have general processing capability, they can house software modules configured to process the pressure data sent by the pressure sensor 40 and dynamically adapt the flush/draw instructions being sent to flow control device 604.

In an embodiment of the present invention, changes in pressure in the fluid column extending through the vascular access device 12 are sensed by the one or more sensors 40. The analyzers 608, 610 are configured to compare the pressure increase to a range of pressures normally expected and/or measured during the initial flush/draw cycles of the integrated sensor system before clots have time to form. Because the fluid flow pressure is a function of catheter length, lumen size, anatomical position and flow rate, the pressure change that forms a threshold for initiating a deviation from the regular draw, flush, calibration cycle is most accurately characterized as a percentage change. If the pressure increase is of sufficient magnitude and/or bears a resemblance to the contours expected from incipient formation of a clot or other occlusion, the analyzers 608, 610 are configured to adapt fluid flow via control commands sent to the flow control device 604 in an attempt to free the clot from the fluid path.

In one embodiment, the flow control device 604 is adapted to interrupt its normal cycle in response to the pressure increase. For example, if in a flushing or calibration cycle, the flow control device 604 is configured to increase flow rate by more quickly advancing roller 802 and pinch point 808 from position 810 to 812, as shown in FIG. 4C. Flow rate can be adjusted to be at the maximum range outlined above for the flush step, such as 10.0 ml/min or even more depending up on the head capability of the flow control device 604. On a percentage basis, a 50% increase is preferred to ensure an adequate flush, but this flush rate could be adjusted in approximately 10% increments (30%, 40%, 60% or 70%), adjusting to expected higher or lower clotting rates or other factors, such as avoiding downtime. The flow rate could be modified dynamically to be proportional to the magnitude or gradient of the increase in pressure detected by the pressure sensor 40. For example, the flow could start with the low 0.25 μl/min described above for the flush step to about 10.0 ml/min in linear increments in proportion to the pressure increase measured by the pressure sensor 40. In this instance, an increasing pressure gradient would induce an increasing acceleration of flow of the solution 602a. Similarly, the flow control device 604 could be configured to respond with repeated, maximum flow rate, high velocity flushes in response to a particularly steep or high increase in pressure in the fluid column. Conversely, if the pressure measured by pressure sensor 40 were to suddenly attenuate, the flow control device 604 could be configured to decelerate movement from position 810 to 812. This latter configuration would avoid flooding the patient with the solution 602a after a clot suddenly breaks free.

In another embodiment, the flow control device 604 could also be configured to increase the length of the flush time. This could be done by adding repeated flushing cycles or slowing the flush rate. The latter option may be less desirable, unless some characteristic of the pressure curve were to indicate that the solvent properties of the solution 602a would have better effect. For example, a sudden, but small, pressure increase might indicate a low-profile partial occlusion by a clot that will have less drag in the solution 602a but has not had time to react to the natural solvent properties of the solution 602a. Such a clot may be more amenable to further time in the solvent. The flow control device 604 in this instance would be configured to slow or stop the advance (FIG. 4C) of the roller 802 from position 810 to 812. Conversely, a slow, progressive increase in the pressure measured by the pressure sensor 40 may indicate that the occlusion has survived several flush cycles and the solvent properties of the solution 602a have not been effective in freeing the clot. Instead, a sudden “water hammer” of high velocity flow induced by the flow control device might mechanically shake such a clot free.

In an instance where the flow control device 604 is in the draw step, the flow control device may be configured to interrupt the draw (such as the draw shown in FIG. 4B), even if no analyte has yet been sensed by analyte sensor 14, in response to selected pressure changes measured by the pressure sensor 40. The flow control device 604 would also be configured to reverse movement of the roller 802 and begin a flush, sending the sample 814 back into the patient. This flush step may be a normal flush as described above, wherein the normal flush is monitored by observing the measured pressures in the fluid column to ensure it is having the desired effect of clearing the clot. The flow control device 604 could be configured to continue its normal cycle thereafter. Or, if the shape or magnitude of the pressure curve meets certain criteria, such as an increase in pressure on a percentage basis of more than 30-80%, the flush profile may be adapted (e.g, length and/or flow rate increased) to more aggressively meet the characteristics of the pressure curve measured by the pressure sensor 40. Alternatively, after reversal, the flow control device 604 may immediately opt for the more aggressive, modulated flush that tracks the characteristics of the pressure monitored by the pressure sensor 40.

In another embodiment, the flow control device 604 may be further configured to dynamically modify the draw step in response to measured pressure changes. For example, the flow control device 604 may be configured to accelerate the draw step by speeding movement of the roller 802 from position 812 to 810, as shown in FIG. 4B. This should reduce the time the blood is within the catheter 12 and hence its tendency to clot. Similar to the flush step, the draw step could be correlated to the characteristics of the pressure curve measured by the pressure sensor 40. For example, the flow control device 604 could be configured to increase the draw rate and/or decrease the time over which draw occurs in proportion to the pressure changes sensed by the pressure sensor 40. The draw rate could be adjusted outside of its normal preferred range, such as by using the 2 ml draw rate described above when normal operation is at 0.085 ml/min to about 0.2 ml/min.

The full capable range of the flow control device 604 on draw, e.g., 0.001 ml/min or less to about 2.0 ml/min for the above-described embodiment, could be mapped to the expected range of pressure changes expected to be sensed by the pressure sensor 40, so that a pressure increase is met with a proportional decrease in draw rate. The draw rate changes could also be non-linear, such that a quick spike in sensed pressure (even if not a maximum pressure reading) results in an immediate increase to a minimum draw rate, such as the 0.001 ml/min flow rate or an entire stop of the draw and reversal to a flush as described above.

Historical pressure data from the pressure sensor 40 could also be stored in memory associated with the catheter 12 or the analyzers 608, 610 or the flow control device 604. This historical data could aid in dynamic control of the flush or draw of the system 600. For example, weighted averaging could be used that takes into account previous pressure sensor measured values and their rate of increase.

In another embodiment, the pressure sensor 40 could also be used to sense the results of a quick perturbation of the fluid column by the flow control device 604. For example, the flow control device could send a very short pulse of solution 602a through to catheter 12 resulting in a high frequency vibration being sent back up the column by a clot. The analyzers 608, 610 could be configured to analyze the spectral frequency of the returned vibration and correlate it to a database of known responses for different types and locations of clots. This could inform whether to reverse into flush mode or how to dynamically modify the flush/draw rates and times to compensate or clear the clot.

In yet another embodiment, wherein the multiple pressure sensors 40 are employed at different locations within the catheter 12, fluid coupler 20, tubing 606 (i.e., along the fluid column from the catheter orifice 12b to the roller 802), the location of the clot may be determined by finding the pair of pressure sensors 40 between which the measured pressure jumps. This may also inform troubleshooting when dynamic fluid control fails to free or clear the clot or other obstruction. For example, the healthcare worker may know that the fluid coupler 20 is occluded and, only it and not the entire catheter 12, needs to be removed and inspected or cleared manually.

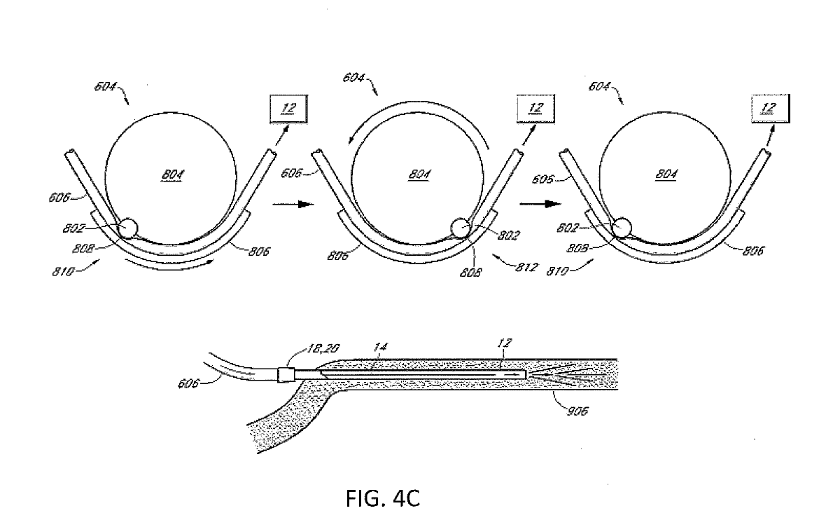

In another embodiment, the present system includes a method and computer program product for detecting and clearing clots, as shown in FIG. 5. The computer program product includes a computer-readable storage medium, such as the non-volatile storage medium, and computer-readable program code portions, such as a series of computer instructions, embodied in the computer-readable storage medium. Typically, the computer program is stored by a memory device, such as memory and executed by an associated processing element, such as processor.

In this regard, FIG. 5 is a flowchart of a method and computer program product according to exemplary embodiments of the invention. In a normal cycle as described in greater detail hereinabove, the integrated sensor system 600 starts a blood draw 300, drawing the blood into the catheter lumen 12a. When the blood reaches the analyte sensor 14, any analyte in the sample 814 is sensed. Then, the flow control device 604 returns the sample 814 into the patient's vasculature in a flush 320. The solution 602a in this embodiment includes a calibration fluid which is used to calibrate 330 the analyte sensor 14. The process is then repeated as long as desired, wherein the patient's blood parameter is continuously sensed over a desired period of time.

During steps 300-330, the integrated sensor system 600 is continuously monitoring the pressure sensor 40 for a characteristic pressure change 340. A characteristic pressure change is any pressure or flow change in the vascular access device 12 that indicates some incipient or existing clot or other occlusion. If this occurs, the parameters of the characteristic pressure change 340 result in one or more of ceasing normal operation and increasing draw rate or reducing draw time 350, starting a flush 360 or increasing an existing flush rate or time 370, as described in more detail above.

It will be understood that each step of the flowchart of FIG. 5, and combinations of the steps in the flowchart, can be implemented by computer program instructions. These computer program instructions may be loaded onto a computer or other programmable apparatus to produce a machine, such that the instructions which execute on the computer or other programmable apparatus create means for implementing the functions specified in the flowchart step(s). These computer program instructions may also be stored in a computer-readable memory that can direct a computer or other programmable apparatus to function in a particular manner, such that the instructions stored in the computer-readable memory produce an article of manufacture including instruction means which implement the function specified in the flowchart step(s). The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions specified in the flowchart step(s).

Accordingly, steps of the flowchart support combinations of means for performing the specified functions, combinations of steps for performing the specified functions and program instruction means for performing the specified functions. It will also be understood that each step of the flowchart, and combinations of steps in the flowchart, can be implemented by special purpose hardware-based computer systems which perform the specified functions or steps, or combinations of special purpose hardware and computer instructions.

The integrated sensor system 600 of the preferred embodiments provides several advantages over prior art technology. Namely, in preferred embodiments, continuous analyte monitoring is enabled. When the analyte is glucose, continuous glucose monitoring enables tight glucose control, which can lead to reduced morbidity and mortality among diabetic hosts. Additionally, the medical staff is not unduly burdened by additional patient interaction requirements. Advantageously, there is no net sample (e.g., blood) loss for the host, which is a critical feature in some clinical settings. For example, in a neonatal intensive care unit, the host is extremely small and loss of even a few milliliters of blood can be life threatening. Furthermore, returning the body fluid sample to the host, instead of delivering to a waste container greatly reduces the accumulation of biohazardous waste that requires special disposal procedures. The integrated sensor system components, as well as their use in conjunction with an indwelling analyte sensor, are discussed in greater detail below.

The preferred embodiments provide several advantages over prior art devices. Advantageously, the movement of the solution 602a and sample occur at a metered rate and are unaffected by changes in head pressure, such as but not limited to when the host elevates his arm or gets up to move around. Also, sample loss to the host is minimized, first by returning all collected samples to the host; and second by substantially preventing back-flow from the host (e.g., into the tubing or reservoir) with a “hard stop” (e.g., a point beyond which the flow control device cannot move fluid into or out of the host). For example, in one preferred embodiment, the flow control device can be configured to deliver no more than 25-ml of solution to the host per hour. In another exemplary embodiment, the flow control device can be configured to draw back no more than 100 μl of blood at any time. Advantageously, the flow rate of solution 602a and sample 814 is carefully controlled, such that both the sample 814 and the solution 602a remain substantially undiluted. Additionally, the solution 602a warms to the host's local body temperature, such that the integrated sensor system 600 is substantially unaffected by temperature coefficient and sensor 14 accuracy is increased.

Despite the above-described advantages of delivering flow at a metered rate, embodiments of the present invention allow the flow control device 604 to override normal operation when the pressure sensor 40 senses an incipient or existing clot or other occlusion. Advantageously, the integrated sensor system 600 can respond to this occurrence with a sudden flush, increased flush and/or an increased draw that reduces draw time. This enables the use of flush solutions 602a that are less invasive than those heavily laden with heparin or other anticoagulants as the draw and flush are actively modulated to respond to clots and other occlusions.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A system for sensing a parameter of blood in a patient's vasculature, the system comprising:
a vascular access device configured for connection to the patient's vasculature and defining an internal fluid passageway configured for fluid communication with the blood;
at least one parameter sensor supported by the vascular access device and configured to sense the blood parameter;
at least one pressure sensor supported in the internal fluid passageway of the vascular access device; and
a flow control device connected in fluid communication with the internal fluid passageway and connected in communication with the at least one pressure sensor,
wherein the flow control device is configured to adapt fluid flow within the internal fluid passageway based on communication with the at least one pressure sensor.

2. A system of claim 1, wherein the flow control device is further configured to draw blood into the internal fluid passageway of the vascular access device and over the parameter sensor.

3. A system of claim 2, wherein the flow control device is further configured to flush a solution over the parameter sensor.

4. A system of claim 3, wherein the flow control device is configured to adapt a draw time for the blood based on communication with the at least one pressure sensor.

5. A system of claim 4, wherein the flow control device is configured to adapt a flush time for the solution based on communication with the at least one pressure sensor.

6. A system of claim 4, wherein the flow control device is configured to temporarily reduce a draw time in response to an increased pressure communicated by the at least one pressure sensor.

7. A system of claim 3, wherein the flow control device is configured to adapt a flow rate for the blood based on communication with the at least one pressure sensor,

8. A system of claim 7, wherein the flow control device is configured to adapt a flush rate for the solution based on communication with the at least one pressure sensor.

9. A system of claim 7, wherein the flow control device is configured to increase the flush rate of the solution in response to detection of a pressure increase by the pressure sensor.

10. A system of claim 9, wherein the flow control device is configured to stop drawing blood and start flushing solution in response to detection of the pressure increase.

11. A system of claim 1, wherein the flow control device is connected in communication with the at least one parameter sensor and is further configured to adapt fluid flow within the internal fluid passageway based on communication with the at least one parameter sensor.

12. A system of claim 1, wherein the vascular access device comprises a catheter defining at least a portion of the internal fluid passageway.

13. A system of claim 12, wherein at least a portion of the parameter sensor is positioned within the lumen of the catheter.

14. A system of claim 1, wherein the vascular access device comprises a hub defining at least a portion of the internal fluid passageway.

15. A system of claim 14, wherein at least a portion of the parameter sensor is positioned within the portion of the internal fluid passageway defined by the hub.

16. A system of claim 3, wherein the flow control device includes a pump and a controller.

17. A system of claim 16, further comprising a reservoir of the solution connected in fluid communication with the internal fluid passageway.

18. A system of claim 17, wherein the reservoir of solution without an anticoagulant component.

19. A system of claim 18, wherein the internal fluid passageway extends from the patient's vasculature and through to a pump of the flow control device and further comprising at least two pressure sensors arrayed along the internal fluid passageway.

20. A system of claim 19, wherein the flow control device is configured to reverse drawing blood into flushing the solution in response to an increased pressure communicated by the at least one pressure sensor.

21. A system of claim 1, wherein the flow control device is further configured to adapt the fluid flow based on a pressure gradient communicated by the at least one pressure sensor.

22. A system of claim 1, wherein the flow control device includes a memory and is capable of storing pressures communicated by the pressure sensor over time and to adapt fluid flow based on a contour of the stored pressures.

23. A method of sensing a parameter of blood in a patient's vasculature, the method comprising:
drawing blood over a parameter sensor and into an internal fluid passageway defined in a vascular access device;
sensing a pressure in the internal fluid passageway;
determining, based on the pressure sensed, that at least a partial occlusion has formed; and
flushing the internal fluid passageway with a solution so as to clear the occlusion.

24. A method of claim 23, wherein the solution without an anticoagulant component.

25. A method of claim 24, wherein flushing with the solution is with a sufficient duration and flow rate to clear the occlusion without an anticoagulant component.

26. A method of claim 23, wherein the occlusion is within the internal fluid passageway.

27. A method of claim 23, wherein the occlusion is on the parameter sensor.

28. A method of claim 23, further comprising sensing a blood parameter after drawing blood over the parameter sensor.

29. A method of claim 28, further comprising flushing the parameter sensor with a calibrant solution and calibrating the parameter sensor before drawing blood over the parameter sensor.

30. A method of claim 29, further comprising repeatedly flushing the parameter sensor with the calibrant, calibrating the parameter sensor, drawing the blood over the parameter sensor and sensing the blood parameter.

31. A method of claim 30, wherein flushing the parameter sensor, calibrating, drawing and sensing are done continuously in repeating series until determining, based on the pressure sensed, that the occlusion has formed.

32. A method of claim 31, further comprising reversing flow within the internal fluid passageway by stopping drawing and starting flushing in response determining that the occlusion has formed.

33. A method of claim 32, further comprising restarting: flushing the parameter sensor, calibrating, drawing and sensing in repeating series after flushing the internal fluid passageway.

34. A method of claim 23, further comprising adapting a draw time for the blood based on sensing the pressure.

35. A method of claim 34, wherein adapting the draw time includes temporarily reducing the draw time in response to sensing an increase in the pressure.

36. A method of claim 23, further comprising adapting a flush rate based on sensing the pressure.

37. A method of claim 36, further comprising increasing a flush rate based on sensing an increase in the pressure.

38. A method of claim 23, wherein sensing the pressure includes sensing a pressure gradient and further comprising adapting the flushing the internal fluid passageway based on the pressure gradient.

39. A method of claim 23, further comprising storing pressures sensed in the internal fluid passageway and adapting the flushing based on a contour of the stored pressures.

40. A fluid-flow optimization system for connecting to a vascular access device connected to a patient's vasculature and supporting at least one parameter sensor and at least one pressure sensor, the system comprising:
a flow control device connected in fluid communication with the internal fluid passageway and connected in communication with the at least one pressure sensor;
wherein the flow control device is configured to adapt fluid flow within the internal fluid passageway based on communication with the at least one pressure sensor.

41. A system of claim 40, wherein the flow control device is further configured to draw blood into the internal fluid passageway of the vascular access device and over the parameter sensor.

42. A system of claim 41, wherein the flow control device is further configured to flush a solution over the parameter sensor.

43. A system of claim 42, wherein the flow control device is configured to adapt a draw time for the blood based on communication with the at least one pressure sensor.

44. A system of claim 43, wherein the flow control device is configured to adapt a flush time for the solution based on communication with the at least one pressure sensor.

45. A system of claim 43, wherein the flow control device is configured to temporarily reduce a draw time in response to an increased pressure communicated by the at least one pressure sensor.

46. A system of claim 42, wherein the flow control device is configured to adapt a flow rate for the blood based on communication with the at least one pressure sensor.

47. A system of claim 46, wherein the flow control device is configured to adapt a flush rate for the solution based on communication with the at least one pressure sensor.

48. A system of claim 46, wherein the flow control device is configured to increase the flush rate of the solution in response to detection of a pressure increase by the pressure sensor.

49. A system of claim 48, wherein the flow control device is configured to stop drawing blood and start flushing solution in response to detection of the pressure increase.

50. A system of claim 40, wherein the flow control device is connected in communication with the at least one parameter sensor and is further configured to adapt fluid flow within the internal fluid passageway based on communication with the at least one parameter sensor.

51. A system of claim 40, wherein the flow control device is configured to reverse drawing blood into flushing the solution in response to an increased pressure communicated by the at least one pressure sensor.

52. A system of claim 40, wherein the flow control device is further configured to adapt the fluid flow based on a pressure gradient communicated by the at least one pressure sensor.

53. A system of claim 40, wherein the flow control device includes a memory and is capable of storing pressures communicated by the pressure sensor over time and to adapt fluid flow based on a contour of the stored pressures.

54. A system of claim 40, wherein the flow control device is further configured to communicate with multiple pressure sensors arrayed along the internal fluid passageway and to adapt the fluid flow based on differences in pressure measured between the sensors.
